(19) United States
(12) Patent Application Publication
(10) Pub. No.: US 2017/0233795 A1
(43) Pub. Date: Aug. 17, 2017

(54) NUCLEIC ACID AMPLIFICATION REAGENT, NUCLEIC ACID AMPLIFICATION CARTRIDGE, AND NUCLEIC ACID AMPLIFICATION METHOD

(71) Applicant: Seiko Epson Corporation, Tokyo (JP)

(72) Inventor: Masayuki UEHARA, Matsumoto (JP)

(21) Appl. No.: 15/428,476

(22) Filed: Feb. 9, 2017

(30) Foreign Application Priority Data
Feb. 15, 2016 (JP) 2016-025613

(51) Int. Cl. C12Q 1/68; G01N 21/78

(52) U.S. Cl. CPC C12Q 1/6818; C12Q 1/686; G01N 21/78

(57) ABSTRACT

A nucleic acid amplification reagent includes a probe which anneals to a target nucleic acid contained in a nucleic acid, and an intercalator which is inserted between base pairs of one strand of the nucleic acid and a complementary strand synthesized on the one strand, and between base pairs of the other strand of the nucleic acid and a complementary strand synthesized on the other strand. The wavelength band of light emitted from the probe and the wavelength band of light emitted from the intercalator at least partially overlap with each other.

This application claims the benefit of Japanese Patent Application No. 2016-025613, filed on Feb. 15, 2016. The content of the aforementioned patent application is incorporated herein by reference in its entirety.

### BACKGROUND

1. Technical Field

The present invention relates to a nucleic acid amplification reagent, a nucleic acid amplification cartridge, and a nucleic acid amplification method.

2. Related Art

A PCR (polymerase chain reaction) method is a technique for amplifying a nucleic acid by repeating a cycle of temperature changes a plurality of times for the nucleic acid utilizing the occurrence of differences in denaturation and annealing of the nucleic acid due to a difference in the chain length of the nucleic acid or the like. By this technique, 2 to the n-th power PCR products (n represents the number of cycles) are obtained.

As a nucleic acid amplification device using such a PCR method, a PCR device disclosed in JP-A-2012-115208 (Patent Document 1) has been proposed by the applicant of the invention. In a biochip mounted in the PCR device disclosed in Patent Document 1, a flow channel through which a reaction mixture containing a target nucleic acid and the like moves is formed, and the reaction mixture is placed in the flow channel, and also a liquid which has a lower specific gravity than that of the reaction mixture and is immiscible with the reaction mixture is filled.

In the PCR device disclosed in Patent Document 1, in the case where a biochip is mounted in a mounting section for mounting the biochip, a heating section which heats a first region of the flow channel formed in the biochip, and a heating section which heats a second region to a temperature which is different for the first region are included. Further, in the PCR device disclosed in Patent Document 1, a drive mechanism which changes the positions of the mounting section and the heating sections between a first position and a second position is included. By this drive mechanism, the reaction mixture in the biochip to be mounted in the mounting section reciprocally moves between the first region and the second region to be heated to different temperatures from each other. According to such a PCR device disclosed in Patent Document 1, the amplification reaction period can be reduced as compared with the case where the temperature of the entire biochip is changed to different temperatures from each other.

However, it was found that there is a case where light is not detected by a light detector even if a target nucleic acid is actually amplified under the conditions that the amplification reaction period is reduced using the PCR device disclosed in Patent Document 1 or the like.

### SUMMARY

An advantage of some aspects of the invention is to facilitate the detection of light by a light detector.

An aspect of the invention is directed to a nucleic acid amplification reagent which is used for amplifying a nucleic acid, and includes a probe which anneals to a target nucleic acid contained in the nucleic acid, and an intercalator which is inserted between base pairs of one strand of the nucleic acid and a complementary strand synthesized on the one strand, and between base pairs of the other strand of the nucleic acid and a complementary strand synthesized on the other strand, wherein the wavelength band of light emitted from the probe and the wavelength band of light emitted from the intercalator at least partially overlap with each other.

In the case of such a nucleic acid amplification reagent, the intensity of light from the probe and the intercalator is increased in a portion where the wavelength bands overlap with each other as compared with a portion where the wavelength bands do not overlap with each other. That is, the target nucleic acid is labeled by supplementing light emission from the probe and light emission from the intercalator with each other.

Therefore, the nucleic acid amplification reagent according to the aspect of invention can increase the amount of light emission for labeling a target nucleic acid as compared with the case where only one of the probe and the intercalator is used, and as a result, the detection of light by the light detector is facilitated.

In the nucleic acid amplification reagent according to the aspect of the invention, it is preferred that the amount of the probe in the nucleic acid amplification reagent is larger than the amount of the intercalator in the nucleic acid amplification reagent.

In the case where the amount of the probe in the nucleic acid amplification reagent is larger than the amount of the intercalator in this manner, the ratio of the intercalator having lower specificity than the probe in the nucleic acid amplification reagent becomes small. Due to this, even in the case where the intercalator is inserted between base pairs of a positive control or a negative control, the probe having higher specificity than the intercalator can specifically bind to the target nucleic acid. Therefore, even in the case where the intercalator is inserted between base pairs of a control, it is possible to ensure that the difference between the amount of light emission for labeling the target nucleic acid and the amount of light emission for labeling a control is a predetermined amount or more.

Another aspect of the invention is directed to a nucleic acid amplification cartridge including a liquid droplet containing the nucleic acid amplification reagent according to the aspect of the invention, and a container having a flow channel through which the liquid droplet moves.

In such a nucleic acid amplification cartridge, the nucleic acid amplification reagent is contained in the liquid droplet. Therefore, the intensity of light from the probe and the intercalator is increased in a portion where the wavelength bands overlap with each other as compared with a portion where the wavelength bands do not overlap with each other. That is, the target nucleic acid is labeled by supplementing light emission from the probe and light emission from the intercalator with each other.

Therefore, the nucleic acid amplification cartridge according to the aspect of invention can increase the amount of light emission for labeling a target nucleic acid as compared with the case where only one of the probe and the intercalator is used, and as a result, the detection of light by the light detector is facilitated.

Still another aspect of the invention is directed to a nucleic acid amplification method including a temperature setting step of setting the temperature of a first region of a container in which a liquid droplet containing a template nucleic acid and the nucleic acid amplification reagent according to the aspect of the invention is placed to the denaturation temperature of a target nucleic acid, and also setting the temperature of a second region which is different from the first region to the synthesis temperature of the target nucleic acid, and an amplification step of repeating a cycle to undergo a denaturation stage in which the liquid droplet is moved to the first region and retained there and a synthesis stage in which the liquid droplet is moved to the second region and retained there a plurality of times.

In such a nucleic acid amplification method, the nucleic acid amplification reagent is contained in the liquid droplet, and the denaturation reaction and the synthesis reaction of the target nucleic acid are repeated in the liquid droplet. At this time, the probe in the nucleic acid amplification reagent anneals to the target nucleic acid, and the intercalator in the nucleic acid amplification reagent is inserted between a strand constituting a nucleic acid containing the target nucleic acid and a complementary strand thereto.

The wavelength band of light emitted from this probe and the wavelength band of light emitted from the intercalator at least partially overlap with each other, and therefore, the light intensity is increased as compared with a portion where the wavelength bands do not overlap with each other. That is, the target nucleic acid is labeled by supplementing light emission from the probe and light emission from the intercalator with each other.

Therefore, the nucleic acid amplification method according to the aspect of invention can increase the amount of light emission for labeling a target nucleic acid as compared with the case where only one of the probe and the intercalator is used, and as a result, the detection of light by the light detector is facilitated.

### BRIEF DESCRIPTION OF THE DRAWINGS

The invention will be described with reference to the accompanying drawings, wherein like numbers reference like elements.

FIG. 1 is a view showing a cross section of a nucleic acid amplification cartridge.

FIG. 2 is a schematic view for illustrating primers and probes.

FIG. 3 is a view showing a state where a nucleic acid reagent solution is introduced into a container of a nucleic acid amplification cartridge.

FIG. 4 is a block diagram of a nucleic acid amplification device.

FIG. 5 is a view schematically showing a state of a rotation mechanism.

FIG. 6 is a view showing a state where a nucleic acid amplification cartridge is mounted in a mounting section.

FIG. 7A is a view showing a state (A) of a thermal cycling treatment.

FIG. 7B is a view showing a state (B) of a thermal cycling treatment.

FIG. 7C is a view showing a state (C) of a thermal cycling treatment.

FIG. 7D is a view showing a state (D) of a thermal cycling treatment.

FIG. 8 is a flowchart showing a nucleic acid amplification method.

### DESCRIPTION OF EXEMPLARY EMBODIMENTS

Hereinafter, embodiments for carrying out the invention will be described with reference to the accompanying drawings. The embodiments and the examples described below are provided only for the purpose of facilitating the understanding of the invention and not for the purpose of limiting the invention. The invention may be changed or modified without departing from the gist of the invention.

### (1) Embodiments

As embodiments of the invention, a nucleic acid amplification reagent, a nucleic acid amplification cartridge, and a nucleic acid amplification method will be described. However, for facilitating the understanding, in the description of a nucleic acid amplification cartridge, a nucleic acid amplification reagent to be placed in the nucleic acid amplification cartridge will also be described. In addition, in the description of a nucleic acid amplification device capable of mounting and dismounting a nucleic acid amplification cartridge, a nucleic acid amplification method which is performed by the nucleic acid amplification device will also be described.

### Nucleic Acid Amplification Cartridge

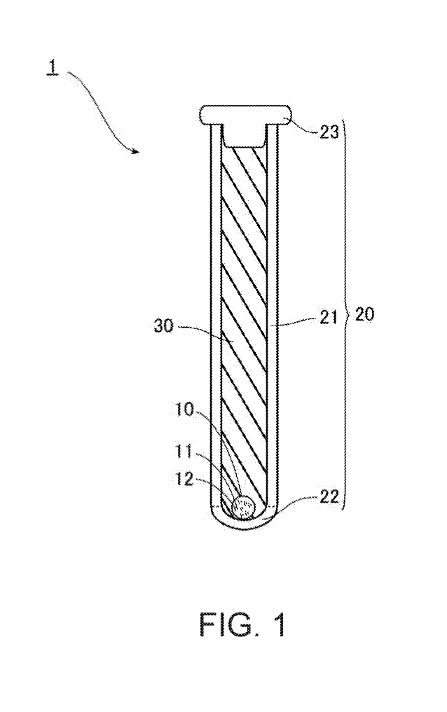

FIG. 1 is a view showing a cross section of a nucleic acid amplification cartridge 1. As shown in FIG. 1, the nucleic acid amplification cartridge 1 can be mounted in and dismounted from a nucleic acid amplification device which amplifies a target nucleic acid in a template nucleic acid, and includes a liquid droplet 10 and a container 20.

The liquid droplet 10 is a place for allowing an amplification reaction of a nucleic acid to proceed, and contains a template nucleic acid 11 and a nucleic acid amplification reagent 12. In FIG. 1, the forms of the template nucleic acid 11 and the nucleic acid amplification reagent 12 are shown conveniently.

The template nucleic acid 11 is a double-stranded nucleic acid extracted from cells derived from a living organism such as a human or a bacterium, virus particles, or the like, and contains a target nucleic acid which is a nucleic acid fragment of an amplification target. Examples of the double-stranded nucleic acid include DNAs (deoxyribonucleic acids) and RNAs (ribonucleic acids).

The nucleic acid amplification reagent 12 is a reagent to be used for amplifying a nucleic acid. This nucleic acid refers to the template nucleic acid 11 or each of a plurality of nucleic acids amplified from the template nucleic acid 11. The nucleic acid amplification reagent 12 mainly includes a primer, a probe, an intercalator, a polymerase, and dNTPs (deoxyribonucleotide triphosphates). In the case where the template nucleic acid 11 is a double-stranded RNA, in order to obtain a cDNA (complementary DNA) of the RNA, a reverse transcriptase, a primer for the reverse transcriptase, and the like are also contained in the nucleic acid amplification reagent 12.

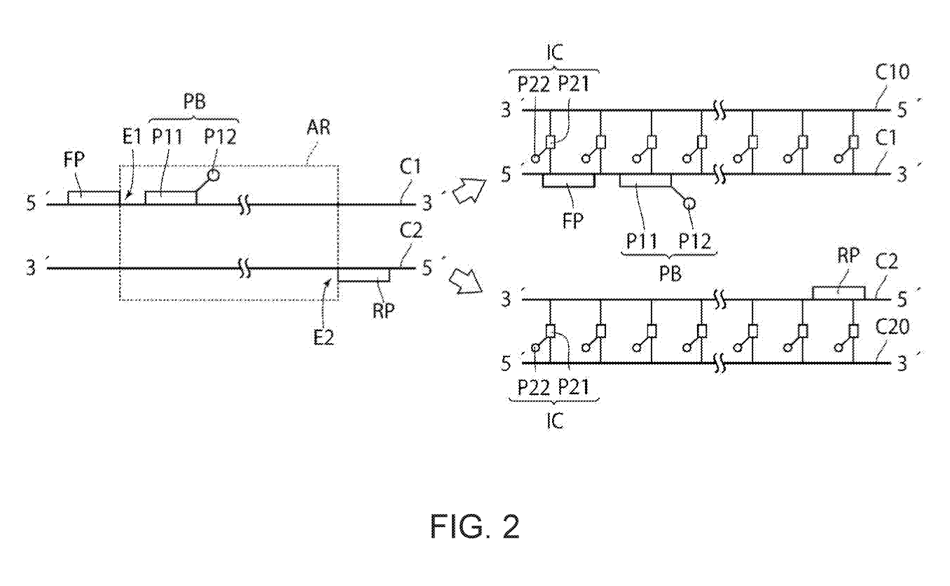

The primer is an oligonucleotide designed so as to anneal to the 3′ end or the 5′ end of the target nucleic acid, and as shown in FIG. 2, a forward primer FP which anneals to part of one strand C1 of a double strand in the nucleic acid and a reverse primer RP which anneals to part of the other strand C2 of the double strand are included.

The probe is a target substance to be used for quantitatively determining the amplification amount of a nucleic acid. Specifically, for example, a TaqMan probe and the like are exemplified. As shown in FIG. 2, a probe PB of this embodiment anneals to a target nucleic acid in a region AR sandwiched between the 3′ end site E1 of the forward primer which anneals to one strand C1 of a nucleic acid and the 3′ end site E2 of the reverse primer which anneals to the other strand C2 of the nucleic acid. As the constituent elements of this probe PB, an annealing section P11 which anneals to the target nucleic acid and a dye P12 to be added to the annealing section P11 are included.

The annealing section P11 has a base sequence which anneals to the target nucleic acid. This base sequence may be a base sequence complementary to the entire base sequence of the target nucleic acid or may be a base sequence complementary to part of the base sequence of the target nucleic acid. Incidentally, the base sequence which specifically anneals to the target nucleic acid in the region AR is preferably used as the base sequence of the annealing section P11. As such a base sequence, for example, an artificial nucleic acid such as a PNA (peptide nucleic acid), an LNA (locked nucleic acid), or an ENA (ethylene bridged nucleic acid) is useful.

The dye P12 is a substance which emits light in a predetermined wavelength band. This dye P12 may be a fluorescent dye or a dye other than a fluorescent dye. Further, the dye P12 may be added to the end of the base sequence of the annealing section P11 or may be added to a site other than the end. In addition, the dye P12 may emit light in a state where it is added to the annealing section P11 or may emit light in a state where it is separated from the annealing section P11.

Examples of the dye which emits light in a state where it is separated from the annealing section P11 include a reporter dye and a quencher dye to be used for a TaqMan probe. The reporter dye and the quencher dye are added to the annealing section P11 such that the light emission of the reporter dye is suppressed by the quencher dye. When the reporter dye is separated from the annealing section P11 by an elongation reaction of a nucleic acid, the suppression of light emission by the quencher dye is released, and therefore, the reporter dye can emit light.

An intercalator is a labeling substance to be used for quantitatively determining the amplification amount of a nucleic acid. Specifically, for example, SYBR Green and the like are exemplified. As shown in FIG. 2, an intercalator IC of this embodiment is inserted between base pairs of one strand C1 of a nucleic acid and a complementary strand C10 synthesized using the strand C1 as a template, and between base pairs of the other strand C2 of the nucleic acid and a complementary strand C20 synthesized using the other strand C2 as a template. As the constituent elements of this intercalator IC, an insertion section P21 which is inserted between base pairs of the template strand C1 and the complementary strand C10 and a dye P22 to be added to a region of the insertion section P21 other than the region are included.

The insertion section P21 is an organic molecule having a planar region which can be inserted between two base pairs constituting a nucleic acid. The dye P22 is a substance which emits light in a predetermined wavelength band, and may be a fluorescent dye or a dye other than a fluorescent dye in the same manner as the dye P12. Further, the dye P22 may emit light in a state where it is added to the insertion section P21 or may emit light in a state where it is separated from the insertion section P21 in the same manner as the dye P12.

In this embodiment, the wavelength band of the light emitted by the dye P12 of the probe PB and the wavelength band of the light emitted by the dye P22 of the intercalator IC at least partially overlap with each other. That is, a portion where the wavelength bands are the same is present in the wavelength band of the light emitted by the dye P12 and in the wavelength band of the light emitted by the dye P22. Incidentally, it is preferred that the wavelength band of the light emitted by the dye P12 and the wavelength band of the light emitted by the dye P22 are the same, however, they may be shifted as long as they have a portion where the wavelength bands even slightly overlap with each other.

In the case where the wavelength band of the light emitted by the dye P12 and the wavelength band of the light emitted by the dye P22 are shifted, it is preferred that an overlapping wavelength band between a wavelength band in a portion where the peak in the spectral distribution of the light emitted by the dye P12 becomes half and a wavelength band in a portion where the peak in the spectral distribution of the light emitted by the dye P22 becomes half is larger.

Further, it is preferred that the peak in the spectral distribution of the light emitted by the dye P12 and the peak in the spectral distribution of the light emitted by the dye P22 are closer to each other. In addition, in the case where each of the dye P12 and the dye P22 is a fluorescent dye, it is preferred that the molecular extinction coefficient and the fluorescence half-life of the dye P12 are closer to those of the dye P22. Further, the chemical structure of the dye P12 and the chemical structure of the dye P22 may be the same or different.

In this embodiment, the amount of the probe PB in the nucleic acid amplification reagent 12 is set to be larger than the amount of the intercalator IC. That is, the concentration of the probe PB in a solution of the nucleic acid amplification reagent 12 is set to be higher than the concentration of the intercalator IC in the solution.

The polymerase is an enzyme which uses a single-stranded nucleic acid as a template and synthesizes a single strand which is a base sequence complementary thereto and the dNTPs are a mixture of four types of deoxyribonucleotide triphosphates (dATP, dCTP, dGTP, and dTTP).

As shown in FIG. 1, the container 20 includes a flow channel section 21 to serve as a flow channel through which the liquid droplet 10 can move, a bottom section 22 which closes an opening on one end side of the flow channel section 21, and a lid section 23 which closes an opening on the other end side of the flow channel section 21. In this embodiment, the flow channel section 21 is formed into, for example, a cylindrical shape, and the bottom section 22 is formed into, for example, a hollow hemispherical shape. Further, the lid section 23 is formed into, for example, a truncated conical shape, and is freely attachable to and detachable from the flow channel section 21.

In this container 20, an oil 30 is placed. The oil 30 has a lower specific gravity than that of a nucleic acid reagent solution to be introduced into the container 20 and is phase-separated from the nucleic acid reagent solution, and for example, 2CS silicone oil, a mineral oil, or the like is used.

The nucleic acid reagent solution is obtained, for example, as follows. That is, a specimen such as cells derived from a living organism such as a human or a bacterium or virus particles is collected with a collecting tool such as a cotton swab, and the template nucleic acid 11 is extracted from the specimen using a known extraction method. Subsequently, by using, for example, a solvent such as water (distilled water or sterile water) or a Tris-EDTA solution (TE), the nucleic acid reagent solution is adjusted in a test tube or the like so that the concentrations of the template nucleic acid 11 and the respective components of the nucleic acid amplification reagent 12 are predetermined values. This nucleic acid reagent solution is introduced into the container 20 using a tool such as a pipette.

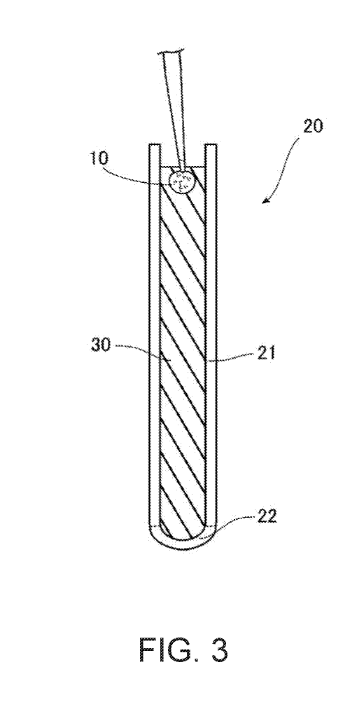

FIG. 3 is a view showing a state where the nucleic acid reagent solution is introduced into the container of the nucleic acid amplification cartridge. As shown in FIG. 3, in the case where the nucleic acid reagent solution is introduced into the container 20, since an action of making the surface area of the interface small acts on the nucleic acid reagent solution, the nucleic acid reagent solution is phase-separated from the oil 30 in the container 20 and therefore is transformed into the liquid droplet 10. The specific gravity of this liquid droplet 10 is higher than that of the oil 30, and therefore, the liquid droplet 10 sinks along the flow channel section 21.

### Nucleic Acid Amplification Device

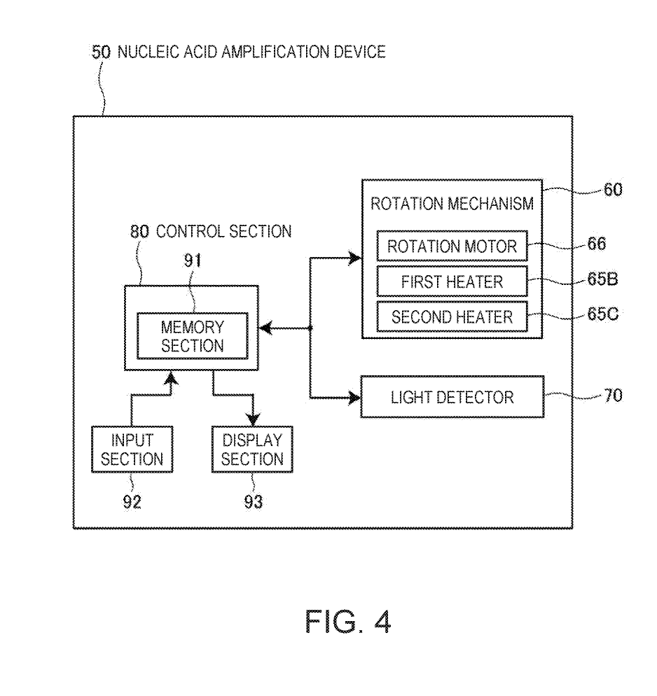

FIG. 4 is a block diagram of a nucleic acid amplification device. As shown in FIG. 4, a nucleic acid amplification device 50 includes a rotation mechanism 60, a light detector 70, and a control section 80.

### Rotation Mechanism

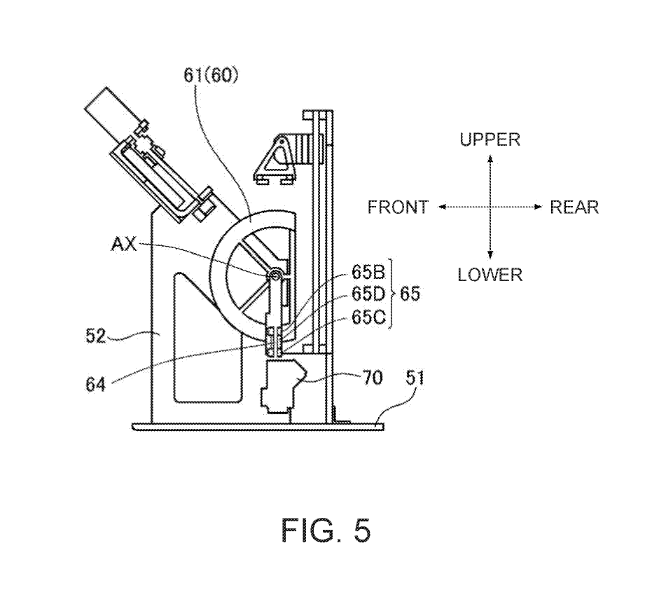

FIG. 5 is a view schematically showing a state of the rotation mechanism. FIG. 5 is a side view of the rotation mechanism 60. In the following description of the nucleic acid amplification device 50, as shown in FIG. 5, upper and lower, front and rear, and right and left are defined. That is, the vertical direction when a base 51 of the nucleic acid amplification device 50 is disposed horizontally is defined as “up and down direction”, and “upper” and “lower” are defined according to the direction of gravity. Further, the axial direction of the rotation axis AX when the nucleic acid amplification cartridge 1 rotates is defined as “right and left direction”, and the direction perpendicular to the up and down direction and the right and left direction is defined as “front and rear direction”.

As shown in FIG. 5, the rotation mechanism 60 includes a rotating body 61 and a rotation motor 66 (FIG. 4) which rotates the rotating body 61. In the rotating body 61, a heater section 65 having an insertion hole 64 capable of mounting and dismounting the nucleic acid amplification cartridge 1 is provided. The rotating body 61 rotates about the rotation axis AX supported by a support table 52 fixed to the base 51 without changing the relative position to the heater section 65 and the nucleic acid amplification cartridge 1 to be mounted in the insertion hole 64 of the heater section 65.

Incidentally, the insertion hole 64 of the heater section 65 in this embodiment functions as a hole through which the nucleic acid amplification cartridge 1 can be put in and out, and also functions as a mounting section for mounting the nucleic acid amplification cartridge 1 put in the hole, however, the hole and the mounting section may be separately provided in the nucleic acid amplification device 50. Further, the number of mounting sections capable of mounting and dismounting the nucleic acid amplification cartridge 1 is not limited to 1, and may be 2 or more.

The rotation motor 66 (FIG. 4) rotates the rotating body 61 according to the instruction from the control section 80 such that the nucleic acid amplification cartridge 1 mounted in the insertion hole 64 of the heater section 65 turns upside down.

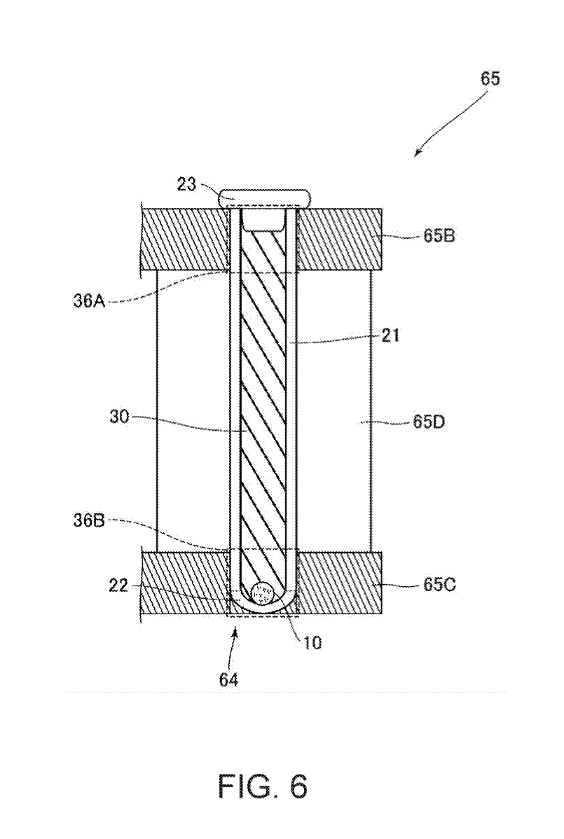

FIG. 6 is a view showing a state where the nucleic acid amplification cartridge is mounted. As shown in FIG. 6, the heater section 65 includes a first heater section 65B for heating a region to a temperature at which a denaturation reaction of the target nucleic acid proceeds and a second heater section 65C for heating a region to a temperature at which a synthesis reaction (an annealing reaction and an elongation reaction) of the target nucleic acid proceeds.

In the case where the nucleic acid amplification cartridge 1 is mounted in the insertion hole 64 of the heater section 65, a first region 36A located on the lid section 23 side of the flow channel section 21 in the container 20 is surrounded by the first heater section 65B. The first heater section 65B heats the first region 36A to a preset temperature in the range of, for example, 95 to 100° C.

Further, in the case where the nucleic acid amplification cartridge 1 is mounted in the insertion hole 64 of the heater section 65, a second region 36B located on the bottom section 22 side of the flow channel section 21 in the container 20 is surrounded by the second heater section 65C. The second heater section 65C heats the second region 36B to a preset temperature in the range of, for example, 50 to 75° C.

In this manner, the first region 36A of the container 20 in the nucleic acid amplification cartridge 1 is heated to a temperature at which the denaturation reaction of the target nucleic acid proceeds, and the second region 36B of the container 20 is heated to a temperature at which the synthesis reaction of the target nucleic acid proceeds.

Incidentally, between the first heater section 65B and the second heater section 65C, a spacer 65D which suppresses heat conduction between the first heater section 65B and the second heater section 65C is disposed. In this spacer 65D, a through-hole is formed at a position along the longitudinal direction of the insertion hole 64 of the first heater section 65B and the second heater section 65C, and the inhibition of insertion of the container 20 of the nucleic acid amplification cartridge 1 in the insertion hole 64 is prevented.

### Light Detector

The light detector 70 is a detector which detects the intensity of light emitted from the liquid droplet 10 placed in the container 20 of the nucleic acid amplification cartridge 1. As shown in FIG. 5, this light detector 70 is disposed, for example, in a state of facing the end of the nucleic acid amplification cartridge 1 mounted in the insertion hole 64 of the heater section 65 spaced apart at a predetermined distance.

The light detector 70 applies light corresponding to the dye P12 of the probe PB and the dye P22 of the intercalator IC according to the detection instruction from the control section 80 and detects the intensity of light emitted by the dyes P12 and P22. More specifically, the intensity of light in a portion where the wavelength band of the light emitted by the dye P12 of the probe PB and the wavelength band of the light emitted by the dye P22 of the intercalator IC overlap with each other is detected. For example, in the case where the dye P12 and the dye P22 are fluorescent dyes having the same chemical structure, excitation light corresponding to the fluorescent dyes is applied thereto, and the fluorescence intensity of the dye P12 and the dye P22 is detected.

Further, the light detector 70 provides data showing the light intensity obtained as the detection result to the control section 80. The light intensity shown by the data reflects the number of times of occurrence of the synthesis reaction (annealing reaction and elongation reaction) of the target nucleic acid. Therefore, it is indicated that as the light intensity shown by the data provided to the control section 80 is higher, the number of target nucleic acids (the number of amplified copies) is larger.

### Control Section

As shown in FIG. 4, the control section 80 includes a memory section 91, and to the control section 80, an input section 92, a display section 93, and the like are connected. In the memory section 91, a region in which a program is stored, a region in which a variety of data such as setting data to be input from the input section 92 and data obtained by a nucleic acid amplification method are stored, and a region in which the program and the data are expanded are included.

The control section 80 appropriately controls the rotation mechanism 60 and the light detector 70 based on the program and the setting data stored in the memory section 91, and a thermal cycling treatment or an amplification analysis treatment is appropriately performed.

### Thermal Cycling Treatment

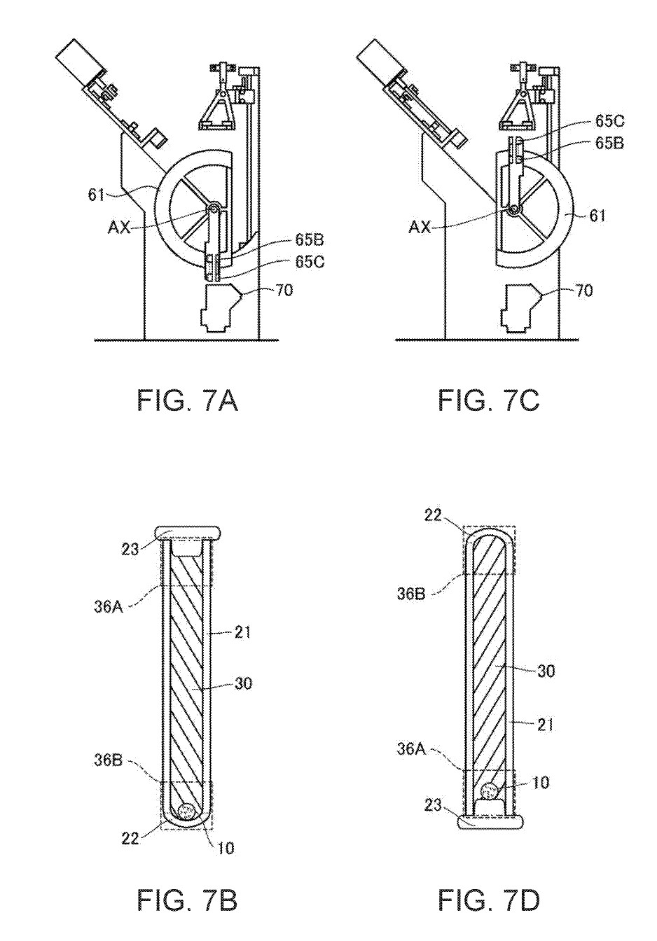

FIGS. 7A to 7D are views showing a state of a thermal cycling treatment. More specifically, FIGS. 7A and 7B show a state of a synthesis stage of a target nucleic acid, and FIGS. 7C and 7D show a state of a denaturation stage of the target nucleic acid.

That is, for example, when receiving a command to perform a thermal cycling treatment from the input section 92, the control section 80 drives the first heater section 65B provided in the rotating body 61, and heats the first region 36A of the container 20 in the nucleic acid amplification cartridge 1 to a temperature at which the denaturation reaction of the target nucleic acid proceeds. Further, the control section 80 drives the second heater section 65C provided in the rotating body 61, and heats the second region 36B of the container 20 in the nucleic acid amplification cartridge 1 to a temperature at which the synthesis reaction of the target nucleic acid proceeds. By doing this, a temperature gradient is formed in the oil 30 filled in the container 20 of the nucleic acid amplification cartridge 1.

It takes a predetermined period from when the first heater section 65B and the second heater section 65C are driven to when the temperature of the oil 30 in the first region 36A has reached, for example, 98° C. and the temperature of the oil 30 in the second region 36B has reached, for example, 54° C. During this period, the amplification reaction of the target nucleic acid does not properly proceed, and therefore, the control section 80 waits during this period as a waiting period.

At this time, as shown in FIGS. 7A and 7B, the rotating body 61 is positioned at a standard position where a portion on the lid section 23 side of the container 20 mounted in the insertion hole 64 of the heater section 65 is disposed on the upper side, and a portion on the bottom section 22 side of the container 20 is disposed on the lower side. In the case where the rotating body 61 is positioned at the standard position, the liquid droplet 10 sinks in the flow channel section 21 by its own weight and is retained in the second region 36B. Therefore, the target nucleic acid contained in the liquid droplet 10 is not transferred to the first round of the denaturation stage.

When the above-mentioned waiting period has elapsed, the control section 80 rotates the rotating body 61 by 180 degrees. In this case, as shown in FIGS. 7C and 7D, the rotating body 61 is positioned at an inverted position where a portion on the lid section 23 side of the container 20 mounted in the insertion hole 64 of the heater section 65 is disposed on the lower side, and a portion on the bottom section 22 side of the container 20 is disposed on the upper side. In the case where the rotating body 61 is positioned at the inverted position, the liquid droplet 10 sinks in the flow channel section 21 by its own weight and moves to the first region 36A. Therefore, the target nucleic acid contained in the liquid droplet 10 is transferred to the denaturation stage.

Further, the control section 80 stops the rotating body 61 only in a denaturation reaction period set as a period of the denaturation stage of the target nucleic acid from when the rotation of the rotating body 61 by 180 degrees is completed (the rotating body 61 is stopped). By doing this, the denaturation reaction of the target nucleic acid contained in the liquid droplet 10 proceeds. Incidentally, the denaturation reaction period is set to at least a period equal to or more than a period in which the liquid droplet 10 moves between the first region 36A and the second region 36B through the flow channel section 21. More specifically, a period of 5 seconds or more and less than 30 seconds is adopted as a general denaturation reaction period, however, a period of 2 seconds or more and less than 5 seconds which is shorter than the general denaturation reaction period may be adopted as the denaturation reaction period.

Subsequently, when the denaturation reaction period has elapsed, the control section 80 changes the position of the rotating body 61 from the inverted position to the standard position by rotating the rotating body 61 by 180 degrees, and as shown in FIG. 7B, the liquid droplet 10 is moved to the second region 36B. By doing this, the target nucleic acid contained in the liquid droplet 10 is transferred to the synthesis stage.

Further, the control section 80 stops the rotating body 61 only in a synthesis reaction period set as a period of the synthesis stage of the target nucleic acid from when the rotation of the rotating body 61 by 180 degrees is completed (the rotating body 61 is stopped). By doing this, the annealing reaction and the elongation reaction of the target nucleic acid contained in the liquid droplet 10 proceed. Incidentally, the synthesis reaction period is set to at least a period equal to or more than a period in which the liquid droplet 10 moves between the first region 36A and the second region 36B through the flow channel section 21 in the same manner as the above-mentioned denaturation reaction period. More specifically, a period of 20 seconds or more and less than 60 seconds is adopted as a general synthesis reaction period, however, a period of 3 seconds or more and less than 20 seconds which is shorter than the general synthesis reaction period may be adopted as the synthesis reaction period.

In this manner, the control section 80 repeats a cycle to undergo the denaturation stage in which the liquid droplet 10 is moved to the first region 36A and retained there and the synthesis stage in which the liquid droplet 10 is moved to the second region 36B and retained there a plurality of times by alternately changing the position between the inverted position and the standard position. The number of cycles to be repeated is set in the control section 80, and for example, set to 50.

### Amplification Analysis Treatment

The amplification analysis treatment is performed in parallel with the thermal cycling treatment at the same time. That is, the control section 80 gives a detection instruction to the light detector 70 for each synthesis reaction period, and stores data showing the light intensity provided from the light detector 70 as the result of the detection instruction in the memory section 91.

As shown in FIGS. 7A and 7B, in the synthesis reaction period, the rotating body 61 is positioned at the standard position, and therefore, the liquid droplet 10 in the container 20 sinks toward the bottom section 22. However, immediately after the rotating body 61 is positioned at the standard position, the liquid droplet 10 has not yet reached the bottom section 22 in some cases. Therefore, the time when the control section 80 gives a detection instruction to the light detector 70 is desirably after a predetermined time has elapsed from when the rotation of the rotating body 61 from the inverted position to the standard position is completed. In particular, it is desirably immediately before the rotating body 61 is rotated from the standard position to the inverted position.

When receiving data showing the light intensity obtained for the number of times which is set as the number of cycles to be repeated, the control section 80 creates an amplification curve showing a change in the light intensity with respect to the number of cycles based on the data obtained for the number of times. When creating the amplification curve, the control section 80 determines acceptance or rejection with respect to the reference amplification efficiency based on the amplification curve, and appropriately causes the display section 93 to display both or either of the determination result and the amplification curve.

### Nucleic Acid Amplification Method

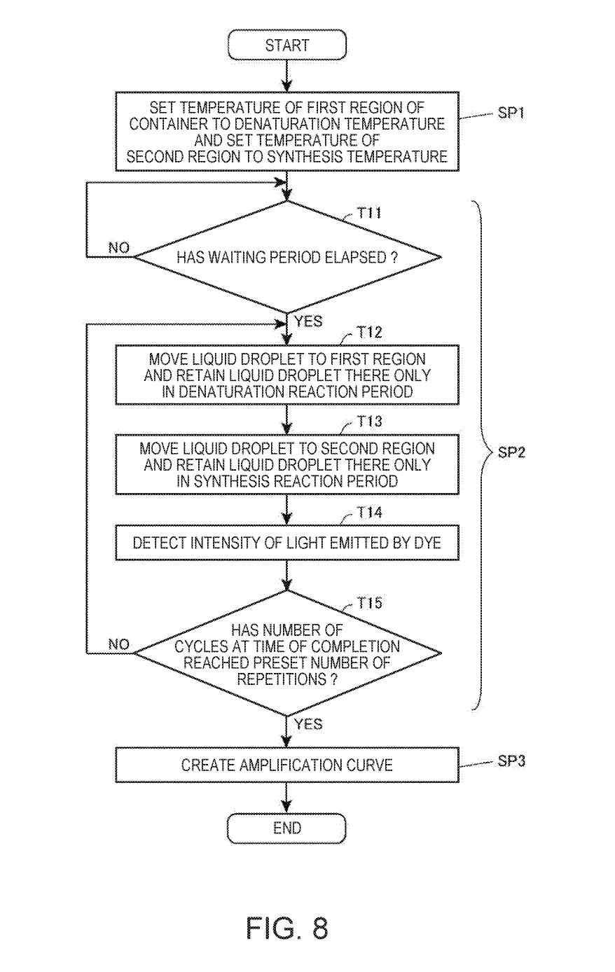

Next, a nucleic acid amplification method which is a procedure of a nucleic acid amplification method and is a series of the above-mentioned respective treatments performed by the control section 80 will be described. FIG. 8 is a flowchart showing the nucleic acid amplification method. As shown in FIG. 8, the control section 80 proceeds to a temperature setting step SP1, and heats the first region 36A of the container 20 in the nucleic acid amplification cartridge 1 to the denaturation temperature set as a temperature at which the denaturation reaction of the target nucleic acid proceeds. Further, the control section 80 heats the second region 36B of the container 20 to the synthesis temperature set as a temperature at which the synthesis reaction of the target nucleic acid proceeds, and then, proceeds to an amplification step SP2.

In a first stage T11 of the amplification step SP2, the control section 80 waits until a waiting period set as a period in which the temperature of a heating target has reached a desired temperature from when heating is started has elapsed, and when the waiting period has elapsed, the control section 80 proceeds to a second stage T12 of the amplification step SP2.

In the second stage T12 of the amplification step SP2, the control section 80 rotates the rotating body 61 from the standard position to the inverted position so that the liquid droplet 10 is moved to the denaturation temperature region (first region 36A) of the container 20. Subsequently, the control section 80 keeps the rotating body 61 stopping so that the liquid droplet 10 is retained in the denaturation temperature region of the container 20 until the denaturation reaction period has elapsed from when the rotating body 61 is positioned at the inverted position. When the denaturation reaction period has elapsed, the control section 80 proceeds to a third stage T13 of the amplification step SP2.

In the third stage T13 of the amplification step SP2, the control section 80 rotates the rotating body 61 from the inverted position to the standard position so that the liquid droplet 10 is moved to the synthesis temperature region (second region 36B) of the container 20. Subsequently, the control section 80 keeps the rotating body 61 stopping so that the liquid droplet 10 is retained in the synthesis temperature region of the container 20 until the synthesis reaction period has elapsed from when the rotating body 61 is positioned at the standard position. When the synthesis reaction period has elapsed, the control section 80 proceeds to a fourth stage T14 of the amplification step SP2.

In the fourth stage T14 of the amplification step SP2, the control section 80 causes the light detector 70 to detect the intensity of light in a portion where the wavelength band of the light emitted by the dye P12 of the probe PB and the wavelength band of the light emitted by the dye P22 of the intercalator IC overlap with each other. Further, when receiving data showing the light intensity as the detection result from the light detector 70, the control section 80 proceeds to a fifth stage T15 of the amplification step SP2.

In the fifth stage T15 of the amplification step SP2, the control section 80 recognizes whether the number of cycles at the time of completion has reached the number of repetitions set as the number of cycles to be repeated. Here, when the number of cycles at the time of completion has not reached the preset number of repetitions, the control section 80 increases the number of cycles at the time of completion by only one, and thereafter returns to the first stage T11 of the amplification step SP2 and repeats the above-mentioned treatments. On the other hand, when the number of cycles at the time of completion has reached the preset number of repetitions, the control section 80 proceeds to an amplification curve creation step SP3.

In the amplification curve creation step SP3, the control section 80 creates an amplification curve using data showing the intensity of light for each number of cycles to be repeated, and the heating of the first region 36A and the second region 36B of the container 20 is stopped. Thereafter, the control section 80 completes the nucleic acid amplification method.

Overview

As described above, the nucleic acid amplification reagent 12 of this embodiment includes a probe PB and an intercalator IC. The probe PB anneals to a target nucleic acid contained in a nucleic acid.

On the other hand, the intercalator IC is inserted between base pairs of one strand C1 of a nucleic acid and a complementary strand C10 synthesized on the strand C1, and between base pairs of the other strand C2 of the of nucleic acid and a complementary strand C20 synthesized on the other strand C2.

The probe PB has a dye P12 and the intercalator IC has a dye P22, and the wavelength band of light emitted from the dye P12 and the wavelength band of light emitted from the dye P22 partially overlap with each other. That is, the wavelength band of light emitted from the probe PB and the wavelength band of light emitted from the intercalator IC partially overlap with each other.

Due to this, the intensity of light in a portion where the wavelength bands overlap with each other is increased as compared with a portion where the wavelength bands do not overlap with each other. That is, the target nucleic acid is labeled by supplementing light emission from the probe PB and light emission from the intercalator IC with each other.

Therefore, the nucleic acid amplification reagent 12 according to this embodiment can increase the amount of light emission for labeling a target nucleic acid as compared with the case where only one of the probe PB and the intercalator IC is used, and as a result, the detection of light by the light detector 70 is facilitated.

Incidentally, in the case where the denaturation reaction period or the synthesis reaction period in the nucleic acid amplification device 50 is shorter than the period generally adopted, the amount of light emission from the probe PB tends to decrease. On the other hand, the intercalator IC has a tendency that the amount of light emission does not decrease as compared with the probe PB even if the denaturation reaction period or the synthesis reaction period is shorter than the period generally adopted. In contrast, the probe PB has a tendency that the specificity for the target nucleic acid is high as compared with the intercalator IC.

In this manner, the nucleic acid amplification reagent 12 of this embodiment includes the probe PB, in which the amount of light emission is likely to vary depending on the change in the denaturation reaction period or the synthesis reaction period, and which has high specificity, and the intercalator IC, in which the amount of light emission is less likely to vary, and which has low specificity. Due to this, in both cases where as the denaturation reaction period or the synthesis reaction period, the general period is adopted, and where a shorter period than the general period is adopted, even if the nucleic acid amplification reagent 12 is not changed, the target nucleic acid can be labeled while maintaining specificity to a certain extent.

In the case where the denaturation reaction period or the synthesis reaction period in the nucleic acid amplification device 50 is shorter than the period generally adopted, even if the amount of light emission from the probe PB having specificity decreases, it can be supplemented by the light emission from the intercalator IC. Therefore, the state where light is almost not detected by the light detector 70 although the target nucleic acid is amplified is suppressed. In this manner, the inclusion of the probe PB and the intercalator IC in the nucleic acid amplification reagent 12 is useful particularly in the case where the denaturation reaction period or the synthesis reaction period in the nucleic acid amplification device 50 is shorter than the period generally adopted.

As described above, the intercalator IC has lower specificity than the probe PB, and therefore, there is a case where the intercalator IC is inserted into base pairs of a positive control or a negative control. In this case, there is a concern that the difference between the amount of light emission for labeling the target nucleic acid and the amount of light emission for labeling a control decreases. On the other hand, in this embodiment, the amount of the probe PB in the nucleic acid amplification reagent 12 is larger than the amount of the intercalator IC in the nucleic acid amplification reagent 12. That is, the ratio of the intercalator IC having lower specificity than the probe PB in the nucleic acid amplification reagent 12 becomes small. Due to this, even in the case where the intercalator is inserted between base pairs of a positive control or a negative control, the probe PB having higher specificity than the intercalator IC can specifically bind to the target nucleic acid. Therefore, even in the case where the intercalator IC is inserted between base pairs of a control, it is possible to ensure that the difference between the amount of light emission for labeling the target nucleic acid and the amount of light emission for labeling a control is a predetermined amount or more.

### (2) Modification Examples

In the above embodiment, the amount of the probe PB in the nucleic acid amplification reagent 12 is set to be larger than the amount of the intercalator IC in the nucleic acid amplification reagent 12. That is, the concentration of the probe PB in a solution of the nucleic acid amplification reagent is set to be higher than the concentration of the intercalator IC in the solution. However, the amount (concentration) of the probe PB in the solution of the nucleic acid amplification reagent 12 may be set to be equal to or smaller than the amount (concentration) of the intercalator IC in the solution. However, in order to ensure that the difference between the amount of light emission for the target nucleic acid and the amount of light emission for a control is a predetermined amount or more even in the case where the intercalator IC is inserted between base pairs of a control, it is preferred that the amount of the probe PB is larger than the amount of the intercalator IC.

Further, in the above embodiment, the amplification reaction is performed in a shorter period than the general denaturation reaction period and synthesis reaction period, however, the amplification reaction may be performed in the general denaturation reaction period and synthesis reaction period.

Further, in the above embodiment, the specific gravity of the liquid droplet 10 is higher than the specific gravity of the oil 30. However, the specific gravity of the liquid droplet 10 may be lower than the specific gravity of the oil 30. Also in this case, the same advantageous effects as those of the above embodiment are obtained.

Further, in the above embodiment, the start time of the denaturation reaction period and the synthesis reaction period is set to the time when the rotation of the rotating body 61 by 180 degrees is completed (the rotating body 61 is stopped), however, the start time may be set to the time when the rotation of the rotating body 61 by 180 degrees starts.

Further, in the above embodiment, the rotation mechanism 60 is adopted as a mechanism for alternately moving the liquid droplet 10 in the container 20 in the nucleic acid amplification cartridge 1 between the first region 36A and the second region 36B. However, any of various drive mechanisms other than the above rotation mechanism 60 can be applied as long as it is a drive mechanism for alternately moving the liquid droplet 10 between the first region 36A which is brought to the denaturation temperature of the target nucleic acid and the second region 36B which is a different region from the first region 36A and is brought to the synthesis temperature of the target nucleic acid in the container 20. Further, a common nucleic acid amplification device which does not have a drive mechanism for alternately moving the liquid droplet 10 may be applied.

Further, in the above embodiment, as the region to which the liquid droplet 10 should be moved in the container 20, the first region 36A which is brought to the denaturation temperature of the target nucleic acid, and the second region 36B which is a different region from the first region 36A and is brought to the synthesis temperature of the target nucleic acid are disposed. However, three regions may be disposed. That is, as the first region 36A, a region which is brought to the denaturation temperature of the target nucleic acid is disposed. Further, as the second region 36B, two regions which are different from each other are disposed, and one region is brought to the annealing temperature set as a temperature at which the annealing reaction in the synthesis reaction of the target nucleic acid proceeds, and the other region is brought to the elongation temperature set as a temperature at which the elongation reaction of the target nucleic acid proceeds. In this manner, the invention is not limited to the above embodiment, in which the temperature changes in one cycle in the following two stages: a denaturation stage and a synthesis stage, and even in the case where the temperature changes in one cycle in the following three stages: a denaturation stage, an annealing stage, and an elongation stage, the liquid droplet 10 can be moved in the container. Incidentally, even in the case where the temperature changes in one cycle in three stages, any of various moving mechanisms other than the rotation mechanism can be applied.

In the above embodiment, the nucleic acid amplification device 50 including the first heater section 65B and the second heater section 65C is applied. However, a nucleic acid amplification device other than the nucleic acid amplification device 50 of the above embodiment may be applied as long as a temperature gradient can be formed inside the container 20.

What is claimed is:

1. A nucleic acid amplification reagent, which is used for amplifying a nucleic acid, comprising:
a probe which anneals to a target nucleic acid contained in the nucleic acid; and
an intercalator which is inserted between base pairs of one strand of the nucleic acid and a complementary strand synthesized on the one strand, and between base pairs of the other strand of the nucleic acid and a complementary strand synthesized on the other strand, wherein
the wavelength band of light emitted from the probe and the wavelength band of light emitted from the intercalator at least partially overlap with each other.

2. The nucleic acid amplification reagent according to claim 1, wherein the amount of the probe in the nucleic acid amplification reagent is larger than the amount of the intercalator in the nucleic acid amplification reagent.

3. A nucleic acid amplification cartridge, comprising:
a liquid droplet containing the nucleic acid amplification reagent according to claim 1; and
a container having a flow channel through which the liquid droplet moves.

4. A nucleic acid amplification method, comprising:
a temperature setting step of setting the temperature of a first region of a container in which a liquid droplet containing a template nucleic acid and the nucleic acid amplification reagent according to claim 1 is placed to the denaturation temperature of a target nucleic acid, and also setting the temperature of a second region which is different from the first region to the synthesis temperature of the target nucleic acid; and
an amplification step of repeating a cycle to undergo a denaturation stage in which the liquid droplet is moved to the first region and retained there and a synthesis stage in which the liquid droplet is moved to the second region and retained there a plurality of times.
